(19) United States
(12) Patent Application Publication
(10) Pub. No.: US 2019/0083532 A1
(43) Pub. Date: Mar. 21, 2019

(54) FORMULATION, APPARATUS, AND METHODS FOR TREATMENT OF BRAIN TRAUMA

(71) Applicant: John C. Hughes, Basalt, CO (US)

(72) Inventor: John C. Hughes, Basalt, CO (US)

(21) Appl. No.: 16/195,428

(22) Filed: Nov. 19, 2018

Related U.S. Application Data
(63) Continuation-in-part of application No. 15/043,573, filed on Feb. 14, 2016, now Pat. No. 10,130,657.
(60) Provisional application No. 62/116,112, filed on Feb. 13, 2015.

(51) Int. Cl. A61K 35/16; A61B 5/0482; A61G 10/02; A61M 5/14; A61M 31/00; A61N 2/00; A61N 5/06; A61K 9/00; A61P 25/00; A61K 33/00; A61K 31/215; A23L 33/12

(52) U.S. Cl. CPC A61K 35/16; A61N 2005/067; A61G 10/026; A61M 5/14; A61M 31/00; A61N 2/006; A61N 5/062; A61K 9/0043; A61P 25/00; A61K 33/00; A61K 31/215; A23L 33/12; A61M 2202/0482; A61M 2202/0415; A61M 2205/051; A61M 2205/057; A61B 5/0482

(57) ABSTRACT

A formulation comprising platelet rich plasma (PRP) for treatment of patients who have experienced brain injury, consisting of: a mixture of human plasma composed of pluripotent adult stem cells comprising very small embryonic like stem cells from autologous plasma or allogeneic plasma, D5W, glutathione, methylcobalamin, and regular insulin. The formulation is infused directly adjacent to a patient's brain through the nostrils or nares of the nasal cavity. Treatment using the formulation may be supplemented with one or more therapies including hyperbaric oxygen therapy (HBOT), cranial osteopathic therapy, intravenous (IV) nutrition, electroencephalographic (EEG) biofeedback, low level laser therapy (LLLT), transcranial magnetic stimulation (TMS), additional PRP pluripotent adult stem cell treatments, and a ketogenic diet and medium-chain triglyceride (MCT) oil therapy.

### CROSS-REFERENCE TO RELATED PATENT APPLICATIONS

This application claims the benefit of U.S. patent application Ser. No. 15/043,573 filed on Feb. 14, 2016 and U.S. Provisional Patent Application No. 62/116,112 filed on Feb. 13, 2015, entitled FORMULATION, APPARATUS, AND METHODS FOR TREATMENT OF BRAIN TRAUMA, the disclosures of which are hereby incorporated herein in their entirety by this reference.

### BACKGROUND OF THE INVENTION

### 1. Field of the Invention

The present invention relates to apparatus and methods for treating personal injuries and, more particularly, to medications, devices, and techniques for treating patients who have suffered brain injury. Specifically, various examples in accordance with the present invention provide a medicinal formulation, devices, and therapeutic protocols developed to treat trauma to the brain of a patient.

### 2. Description of the Prior Art

Incidences of traumatic brain injury in both developed and developing countries are on the rise. The primary reasons for the increase in traumatic brain injury include growth in population and a growing number of traffic accidents and other emergencies such as natural disasters, sports injuries, falls, and assaults. Moreover, modern military conflict has led to an additional steep rise in traumatic brain injury due to blast injuries as well as direct combat-related head injuries. The burden of traumatic brain injury in the USA has been estimated to be 1.5 million cases each year, with an annual economic cost exceeding $56 billion as of 2010.

Traumatic brain injury is characterized by mechanical forces which disrupt brain tissue in addition to all of the destructive inflammatory and ischemic/hypoxic processes of other brain injuries. For example, brain trauma may occur as a result of a force impact to the head of a person. The sources of force impacts vary. Brain trauma can be caused by accidents such as a vehicular collision during which a person's head is thrust against the steering wheel or dash board or the person's head strikes the ground as a consequence of a fall from a ladder. Brain trauma can be caused intentionally such as blunt force applied to a person's head during commission of a criminal assault or during military combat. Brain trauma may also result from any cause that results in acute loss of blood or oxygen to the brain causing brain tissue damage.

Various techniques for the treatment of traumatic brain injury are known. The various techniques include the following.

Hyperbaric oxygen therapy (HBOT) is a known treatment which enhances the body's natural healing process by inhalation of 100% oxygen in a total body chamber, where atmospheric pressure is increased and controlled. HBOT is used for a wide variety of treatments usually as a part of an overall medical care plan. Under normal circumstances, oxygen is transported throughout the body only by red blood cells. With HBOT, oxygen is dissolved into all of the body's fluids, the plasma, the central nervous system fluids, the lymph, and the bone and can be carried to areas where circulation is diminished or blocked. In this way, extra oxygen can reach all of the damaged tissues, so that the body can better support its own healing process. The increased oxygen greatly enhances the ability of white blood cells to kill bacteria, reduces swelling, and allows new blood vessels to grow more rapidly into the affected areas. It is a simple, non-invasive, and painless treatment. When cells in the brain die, either from trauma or lack of oxygen, blood plasma leaks out into surrounding brain tissue causing swelling and reducing blood flow. These otherwise normal cells go dormant because they cannot function without the appropriate amount of oxygen. HBOT dramatically increases the oxygen carried in the blood plasma, making oxygen available to heal damaged capillary walls, preventing plasma leakage, and reducing swelling. As the swelling decreases, blood flow can be restored to the dormant tissue (neovascularization), and these cells then have the potential to function again.

An additional known treatment for traumatic brain injury is intravenous (IV) nutrition therapy. Brain trauma triggers hypermetabolic and catabolic states, severely impairing nitrogen homeostasis. Brain trauma is characterized by disproportional pro-inflammatory cytokine (e.g., tumor necrosis factor-α, interleukin-1, and interleukin-6) production and release that is associated with increased counter-regulatory hormones (e.g., cortisol, glucagon, and catecholamines) release. This process leads to increased systemic and cerebral energy needs, even in paralyzed patients. The increased energy needs can persist for long periods. Nutrition therapy should start early: within 24 to 48 hours of admission to an intensive care unit. The feeding should be adjusted based on the patient's nutritional requirements over the next 48 to 72 hours. This process is often challenging in severe brain trauma patients. The Brain Trauma Foundation recommends that total nutritional support should be achieved within seven days of the injury. Installing enteral access and starting enteral nutrition should be attempted as soon as volume resuscitation is complete and the patient is hemodynamically stable. Early nutritional support is able to reduce the secretion of catabolic hormones, which is already increased in this setting. IV nutrition therapy is also able to at least partially preserve the previous nutritional conditions of the patient, thereby partially preserving body weight and muscle mass. Additionally, IV nutrition therapy results in less intestinal bacterial proliferation and therefore less translocation.

It is also known to treat brain trauma patients using electroencephalographic (EEG) biofeedback therapy. EEG biofeedback is a methodology for harmonizing aberrant or overactive brainwave patterns such that patients can learn to redevelop the most appropriate brainwave pattern for each environmental or social situation. Clinical evidence has shown the effectiveness of EEG biofeedback training as an adjunct modality for remediating the symptoms of minor closed head injury. For example, the Neurofeedback Wellness Center located in The Woodlands, Texas has reported that biofeedback training appears to be effective even years post-injury, when spontaneous remediation is no longer expected.

Another known traumatic brain injury treatment is cranial osteopathic therapy. Cranial osteopathy (also called cranial therapy or craniosacral therapy) is one variety of osteopathic manipulative therapies. Cranial osteopathy stimulates healing by using gentle hand pressure to manipulate the skeleton and connective tissues, especially the skull and sacrum (the large, triangular bone at the base of the spinal column). Cranial osteopathy is based on the espoused theory that the central nervous system, including the brain and spinal cord, has subtle, rhythmic pulsations that are vital to health and can be detected and modified by a skilled practitioner.

Low level laser (light) therapy (LLLT) has been clinically applied for a wide range of medical indications requiring protection from cell and tissue death, stimulation of healing, and repair of injuries, as well as reduction of pain, swelling, and inflammation, and has been investigated in connection with the treatment of traumatic brain injury. Evidence is suggesting that red or near-infra-red light (at wavelengths that can penetrate tissue) is absorbed by mitochondrial chromophores leading to increased cellular respiration, more adenosine triphosphate (ATP) synthesis, modulation of oxidative stress, and nitric oxide production that together lead to activation of signaling pathways and gene transcription.

Transcranial magnetic stimulation (TMS) and in particular repetitive TMS (rTMS) is also known to have been used for treatment of traumatic brain injury. TMS is a painless method to stimulate the human brain. Repeated applications of TMS can influence brain plasticity and cortical reorganization through stimulation-induced alterations in neuronal excitability.

Another known therapy for brain trauma is adult stem cell therapy. Stem cells are undifferentiated, or blank, cells with the potential to give rise to many different cell types that carry out different functions. While the stem cells in adult bone marrow or umbilical cord blood tend to develop into the cells that make up the organ system from which they originated, these multipotent stem cells can be manipulated to take on the characteristics of neural cells. To date, there have been two widely-held views on how stem cells may work to provide potential treatments for brain damage caused by injury or neurodegenerative disorders. One school of thought is that stem cells implanted into the brain directly replace dead or dying cells. The other, more recent view is that transplanted stein cells secrete growth factors that indirectly rescue the injured tissue. A University of Florida study conducted in 2013 concluded that the transplanted stem cells create a neurovascular matrix that bridges the long-distance gap between the region in the brain where host neural stem cells arise and the site of injury. This pathway, or “biobridge,” ferries the newly emerging host cells to the specific place in the brain in need of repair, helping promote functional recovery from traumatic brain injury.

Additionally, it is known that platelets are specialized blood cells that play a critical role in clot formation and injury healing. They are naturally extremely rich in connective tissue growth factors. Activating and injecting these growth factors into damaged ligaments, tendons, and joints ignites a person's body's own stem cells and stimulates the natural repair process.

Platelet rich plasma (PRP) has also been reported to be combined with stem cells that may be mobilized from a patient's bone marrow using the FDA approved drug Neupogen® which prods the marrow to produce replacement stem cells that tend to be more robust than those that were mobilized. The “revitalized” bone marrow (BMAC) may then be harvested and infused by intravenous injection or other means. In addition, growth factor rich PRP from a patient's own blood may be mixed with the BMAC prior to infusion. The growth factors activate stem cells and enhance their activity. The stem cells may be directed to target tissues by the use of special factors that are extracted from a patient's own blood which are injected into those areas (such as joints) where the stem cells need to go in order to effect repair and restoration. One of these factors is SDF-1 which can bring 50 times more stem cells to the injured organ or tissue than is otherwise possible. However, the use of PRP, which is composed of platelet growth factors and pluripotent adult stem cells, has not heretofore been known for treating brain trauma.

A ketogenic diet and medium-chain triglyceride (MCT) oil have been used with autistic and epileptic children. Moreover, there is evidence from uncontrolled clinical trials and studies in animal models that a ketogenic diet can provide symptomatic and disease-modifying activity in a broad range of neurodegenerative disorders including Alzheimer's disease and Parkinson's disease, and may also be protective in instances of traumatic brain injury and stroke. A ketogenic diet is a high fat, adequate protein, low carbohydrate diet. MCT oil, for example, Bulletproof® Brain Octane oil, contains the most ketogenic MCT in coconut oil, C8, from the heart of the coconut. One aspect of the hypothesis for treatment using a ketogenic diet and MCT oil is an associated modification of the tricarboxylic acid cycle to increase the synthesis of the neurotransmitter gammaaminobutyric acid (GABA), leading to neuronal hyperpolarization and less of the neuroxcitatory glutamate. A ketogenic diet and MCT oil provide improved protection from oxidative stress and increased synthesis of calming neurotransmitters (including GABA).

The present invention addresses treatment of brain injury with a novel formulation, apparatus, and methods of treatment. The various examples of the present invention have been demonstrated to provide efficacious treatment of brain injury.

### SUMMARY OF THE INVENTION

In accordance with various non-limiting examples of the present invention, treatment of brain injury comprises a protocol that may include various therapies. The particular therapies are selected to provide optimal recovery of a patient suffering from sub-acute to chronic symptoms related to brain injury.

Considered in more detail, in accordance with a non-limiting example of the present invention, platelet rich plasma (PRP) infusion therapy is administered by a qualified physician to a patient who has experienced brain injury. In accordance with a non-limiting example of the present invention, the PRP infusion comprises a form of blood plasma that is rich in platelets (with over 300 growth factors), cytokines, intact blood cells, progenitor cells, white blood cells, pluripotent adult stem cells, and many other cell signaling molecules. The PRP infusion further preferably comprises the PRP combined with drugs and nutrients. Although the PRP infusion in accordance with non-limiting examples of the present invention preferably comprises a combination of nutrients, the combination of nutrients is not limited to particular nutrients or combinations of nutrients, and nutrients may be added as needed, or different ratios of the same nutrients may comprise the PRP infusion, as long as the PRP remains a plasma-based cocktail. The PRP infusion is preferably infused directly adjacent to the brain through the nostrils or nares of the nasal cavity. Additionally, in accordance with the present invention, a formulation delivery apparatus is provided for delivering the intranasal PRP infusion. In accordance with a non-limiting example, the patient may be treated using PRP infusion therapy at the initial consultation and at least an additional consultation or as frequently as every three weeks for a total of three treatments over six weeks. In one non-limiting example, the PRP infusion therapy may be continued every four months for the following year.

In accordance with an additional non-limiting example of the present invention, a regimen of one or more treatments using hyperbaric oxygen therapy (HBOT) is administered by a qualified physician to a patient who has experienced traumatic brain injury. During the period of administration of the HBOT treatments, the patient is also treated using PRP infusion one or more times. Additionally, following each treatment of PRP infusion, cranial osteopathic therapy is preferably performed on the patient.

In accordance with a further non-limiting example of the present invention, a regimen of 40 treatments using hyperbaric oxygen therapy (HBOT) is administered by a qualified physician to a patient, who has experienced traumatic brain injury, over a period of six weeks. During the six-week period of administration of the HBOT treatments, the patient is treated using PRP infusion at the time of the first HBOT treatment, at three weeks, and also at six weeks. Following each treatment using PRP infusion initially and again at three and six weeks, cranial osteopathic therapy is performed on the patient.

In accordance with another non-limiting example of the present invention, a regimen of 40 initial treatments using hyperbaric oxygen therapy (HBOT) administered by a qualified physician to a patient, who has experienced traumatic brain injury, over a period of six weeks is supplemented by two follow-up regimens of 20 treatments using HBOT at three and nine months from the initial HBOT regimen. The patient is treated using supplemental PRP infusions at four-month intervals for a year from the time that the initial HBOT treatment is administered. Following each supplemental treatment using PRP infusion initially and again at four-month intervals for a year from the time that the initial HBOT treatment is administered, cranial osteopathic therapy is performed on the patient.

In accordance with additional non-limiting examples of the present invention, PRP infusion, HBOT, and cranial osteopathic therapy are further supplemented with additional therapies. The additional supplemental therapies may include one or more of the following therapies: intravenous (IV) nutrition, electroencephalographic (EEG) biofeedback, low level laser therapy (LLLT), and transcranial magnetic stimulation (TMS) treatments.

In accordance with the various examples of the present invention, treatment of brain injury using PRP infusion has demonstrated efficacy in the treatment of brain injury. The therapeutic effects can also be enhanced when coordinated with other treatments.

The time periods between administrations of the various treatments and the durations of treatments comprising protocols in accordance with the non-limiting examples of the present invention are examples only and are not limited to particular frequencies or lengths of time that particular treatments are administered. Furthermore, the administration of treatments comprising a protocol in accordance with the non-limiting examples of the present invention are examples only and are not limited to a particular sequence.

The foregoing and other objects, features, and advantages of the present invention will become more readily apparent from the following detailed description of various examples of the present invention, which proceeds with reference to the accompanying drawing.

### BRIEF DESCRIPTION OF THE DRAWING

The various examples of the present invention will be described in conjunction with the accompanying figures of the drawing to facilitate an understanding of the present invention. In the figures, like reference numerals refer to like elements. In the drawing:

FIG. 1 illustrates the anatomy of a nostril (or naris) which is one of the two channels of the nose, from the point where they bifurcate to the external opening.

FIG. 2 illustrates additional anatomical elements of the nasal cavity.

FIG. 3 is an elevational view of a nasal atomizer with a pipette extension for infusion of a formulation comprising platelet rich plasma infusion into a nostril(s) in accordance with an example embodiment of the present invention.

FIG. 4 is a flowchart of an example therapeutic treatment in accordance with an example embodiment of a method in accordance with the present invention.

FIG. 5 is an elevational view of a nasal atomizer with a catheter extension for infusion of a formulation comprising platelet rich plasma into a nostril(s) in accordance with another example embodiment of the present invention.

FIG. 6 is an elevational view of a syringe with a catheter extension for infusion of a formulation comprising platelet rich plasma into a nostril(s) in accordance with a further example embodiment of the present invention.

### DETAILED DESCRIPTION OF THE PREFERRED EXAMPLE EMBODIMENTS

In accordance with various non-limiting examples of the present invention, protocols are provided to repair injured brain tissue in patients suffering from sub-acute to chronic symptoms related to traumatic brain injury. The traumatic brain injury protocols consist of one or more therapies. The traumatic brain injury protocols have been proven to aid in the recovery of patients who have suffered traumatic brain trauma caused by automobile injury, blunt trauma injury, military injury, or sports injury, for example, as well as anoxic or chemical/toxic brain injuries. The traumatic brain injury protocols have also been employed to treat neurodegenerative conditions such as Alzheimer's disease, Parkinson's disease, multiple sclerosis (MS), and cerebral or cerebellar atropy, as well as neurovascular conditions such as embolic stroke. The traumatic brain injury protocols have also been proven to aid in brain enhancement (including memory or cognitive enhancement), as well as in brain longevity treatment.

Human plasma is composed of various components including but not limited to: very small embryonic like stem cells from autologous plasma or allogeneic plasma (VSELs). These pluripotent VSELs are also known as blastomere like stem cells, or lineage-uncommitted stem cells, and coexist with hematopoietic stem cells which are lineage-committed stem cells in human plasma, which comprise PRP. Only the lineage-uncommitted stem cells are pluripotent, which means that they have the ability to form all adult cell types and are thus legitimate for homologous use in all tissues of the human body. In accordance with a non-limiting example of the present invention, a formulation comprising platelet rich plasma (PRP) comprising pluripotent adult stem cells is used in the treatment of patients who have experienced traumatic brain injury. The PRP is a specialized form of human plasma that is rich in growth factors, cytokines, and pluripotent adult stem cells.

The formulation in accordance with one non-limiting example of the present invention also comprises drugs and nutrients for treatment of traumatic brain injury. In accordance with one non-limiting example, the formulation consists of a mixture of approximately 75% by volume PRP comprising pluripotent adult stem cells, 15% by volume D5W, 5% by volume glutathione, 4% by volume methylcobalamin, and less than 1% by volume regular insulin. The foregoing volumetric percentages are approximate percentages in that the percentages have a range of plus or minus 1%.

After the above-described formulation is prepared, in accordance with a non-limiting example of the present invention, the formulation is infused slowly into the frontal region of the brain of a patient suffering from traumatic brain injury. In accordance with one non-limiting example, the formulation is delivered directly adjacent to the brain through the nostrils or nares of the nasal cavity.

In accordance with another non-limiting example, the human plasma, including plasma based pluripotent adult stem cells, is mixed with activated PRP, growth factors and combined with a nutrient known as nicotinamide adenine dinucleotide+ (NAD+) for a second intranasal infusion. In accordance with a further non-limiting example, within 24 hours after receiving the first PRP infusion, the second formulation composed of activated PRP containing its concentrated lineage-committed and lineage-uncommitted stem cells combined with NAD+ is infused intranasally and intravenously. The formulations in accordance with the non-limiting examples comprising NAD+ are activated by ozone and a green laser light at a specific frequency.

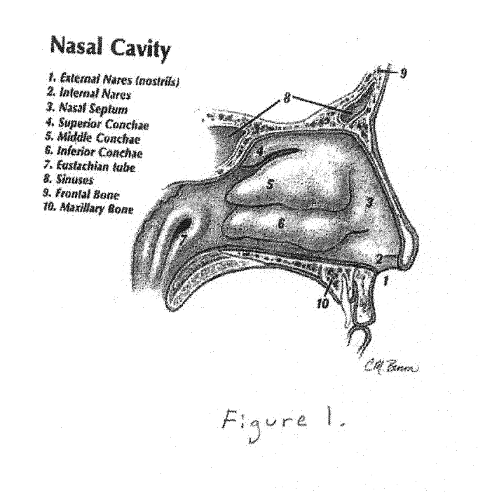

FIG. 1 illustrates the anatomy of a nasal cavity including a nostril (or naris) which is one of the two channels of the nose, from the point where they bifurcate to the external opening indicated by the numeral 1. The internal naris is indicated by the numeral 2. The septum that divides the nose into two nares is indicated by the numeral 3. The internal tissue of the nasal cavity comprises superior conchae, middle conchae, and inferior conchae indicated by the numerals 4, 5, and 6, respectively.

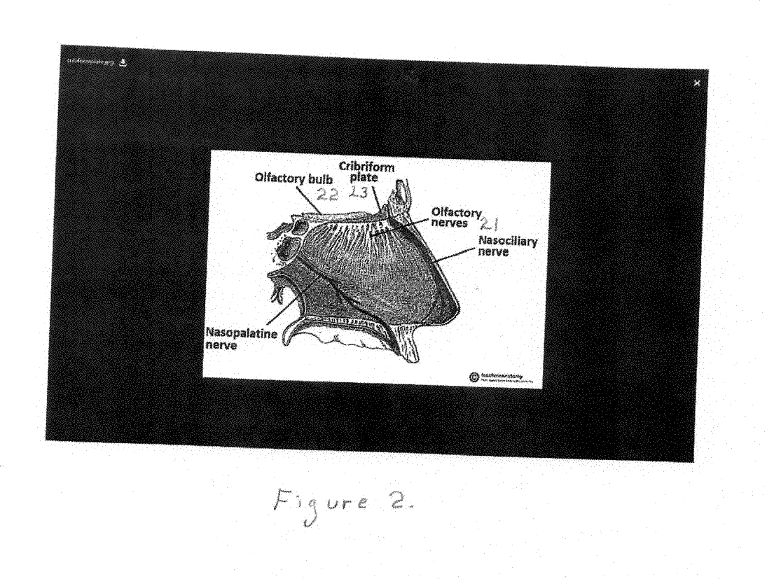

Additional anatomical elements of the nasal cavity appear in FIG. 2. These anatomical elements include the olfactory nerves and the olfactory bulb indicated by the numerals 21 and 22, respectively. The olfactory bulb 22 is on the inferior (bottom) side of the brain.

In accordance with a non-limiting example of the present invention, the formulation described above is delivered slowly through the nares 2 shown in FIG. 1 and infused into the inferior side of the brain of a patient suffering from traumatic brain injury by means of a delivery apparatus. Non-limiting examples of the apparatus for delivering the formulation will now be described.

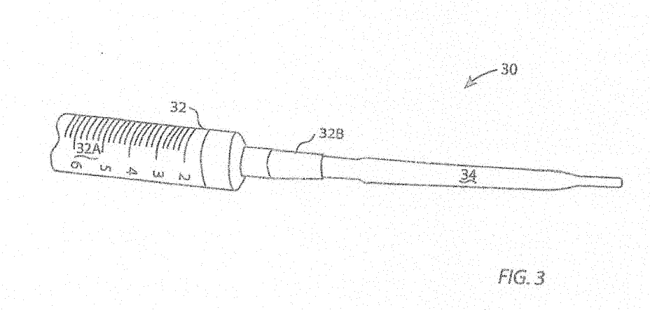

A non-limiting example of the formulation delivery apparatus in accordance with the present invention is shown in FIG. 3, generally indicated by the numeral 30. One non-limiting example of the delivery apparatus is a nasal atomizer comprising a 10 cc syringe 32 having a reservoir 32A and an atomizing dispensing tip 32B. The formulation delivery apparatus 30 further comprises a single-use pipette tip 34 selectively attached to the dispensing tip 32B of the syringe 32.

In order to administer the formulation to the patient suffering from traumatic brain injury, the pipette tip 34 is slid onto the dispensing tip 32B of the syringe 32. The reservoir of the syringe 32 is filled with 10 cc of the formulation to be administered, such as the formulation described above.

The pipette tip 34 is then inserted into a naris 1, 2 of the traumatic brain injury patient, and the syringe 32 is actuated to provide a nasal atomizer with the pipette tip 34 being positioned in the nasal cavity to provide delivery of the formulation to the fossa of the cribiform plate 23 shown in FIG. 2 under the nasal bridge just posterior to the frontal bone of the cranium to infuse the formulation into the patient's brain.

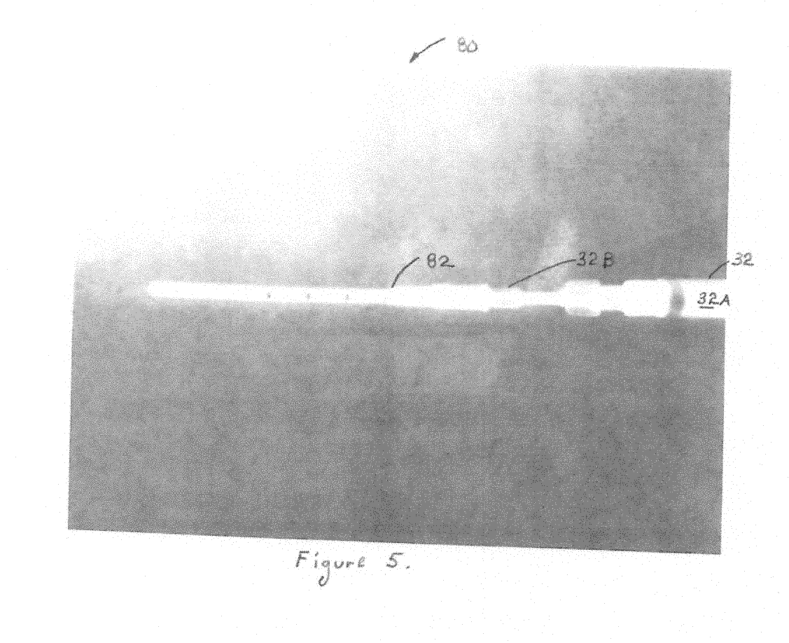

Another non-limiting example of the formulation delivery apparatus in accordance with the present invention is shown in FIG. 5, generally indicated by the numeral 80. One non-limiting example of the delivery apparatus is a nasal atomizer comprising a 10 cc syringe 32 having a reservoir 32A and an atomizing dispensing tip 32B. The formulation delivery apparatus 80 further comprises a catheter 82 selectively attached to the atomizing dispensing tip 32B of the syringe 32.

In order to administer the formulation to the patient suffering from traumatic brain injury, the catheter 82 is slid onto the atomizing dispensing tip 32B of the syringe 32. The reservoir of the syringe 32 is filled with 10 cc of the formulation to be administered, such as the formulation described above.

The catheter 82 is then inserted into a naris 2 of the traumatic brain injury patient, and the syringe 32 is actuated to provide a nasal atomizer with the catheter being positioned in the nasal cavity to provide delivery of the formulation to the fossa of the cribiform plate 23 shown in FIG. 2 under the nasal bridge just posterior to the frontal bone of the cranium to infuse the formulation into the patient's brain.

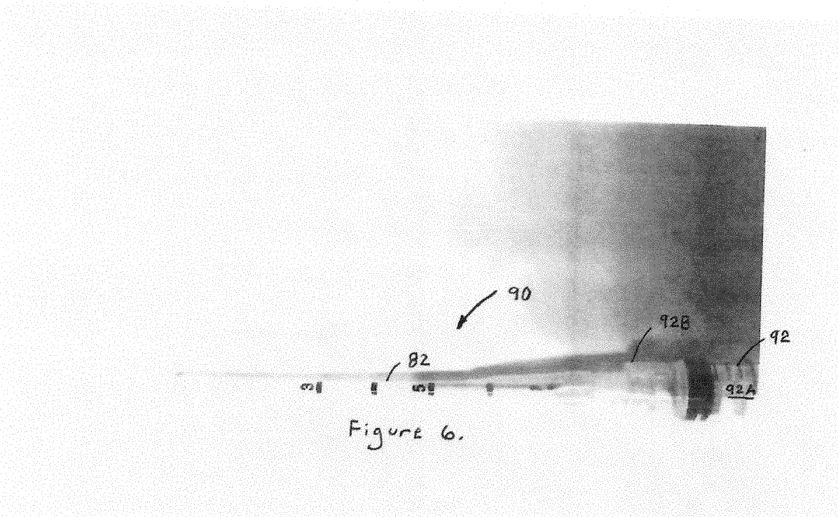

A further non-limiting example of the formulation delivery apparatus in accordance with the present invention is shown in FIG. 6, generally indicated by the numeral 90. One non-limiting example of the delivery apparatus is a 10 cc syringe 92 having a reservoir 92A and a dispensing tip 92B. The formulation delivery apparatus 90 further comprises a catheter 82 selectively attached to the dispensing tip 92B of the syringe 92.

In order to administer the formulation to the patient suffering from traumatic brain injury, the catheter 82 is slid onto the dispensing tip 92B of the syringe 92. The reservoir of the syringe 92 is filled with 10 cc of the formulation to be administered, such as the formulation described above.

The catheter 82 is then inserted into a naris 2 of the traumatic brain injury patient, and the syringe 92 is actuated with the catheter being positioned in the nasal cavity to provide delivery of the formulation to the fossa of the cribiform plate 23 shown in FIG. 2 under the nasal bridge just posterior to the frontal bone of the cranium to infuse the formulation into the patient's brain.

The novel intranasal delivery device in the form of the catheter 82 attached to a syringe is superior to other intranasal devices, because the device extends much farther into the upper nares and closer to the brain to deliver a higher concentration of the human plasma (and all of its components) directly to the brain. In contrast, known devices including aerosols, metered dose inhalers, nebulizers, sprays, or even creams, provide only a small fraction to the brain, because a portion of the substance remains in the lower nares or in the skin.

Preferably, a novel delivery method involves having a patient lie on a tilted or flat procedure table with his or her head and neck in full extension, with his or her nose completely inverted and perpendicular to the floor. The patient's knees (and, preferably, back) are also elevated above the head and neck. Using the novel intranasal delivery device in the form of the catheter 82 attached to a syringe, the human plasma and all of its components are delivered directly to the patient's upper nares bilaterally. The patient is maintained in the knee-raised, neck-extended position for a minimum of 10 minutes and a maximum of 20 minutes. This novel delivery method is superior to known intranasal delivery methods in which patients are placed in an upright position without the head titled fully back. An upright position does not allow for the maximum amount of human plasma and all of its components to be delivered to the brain.

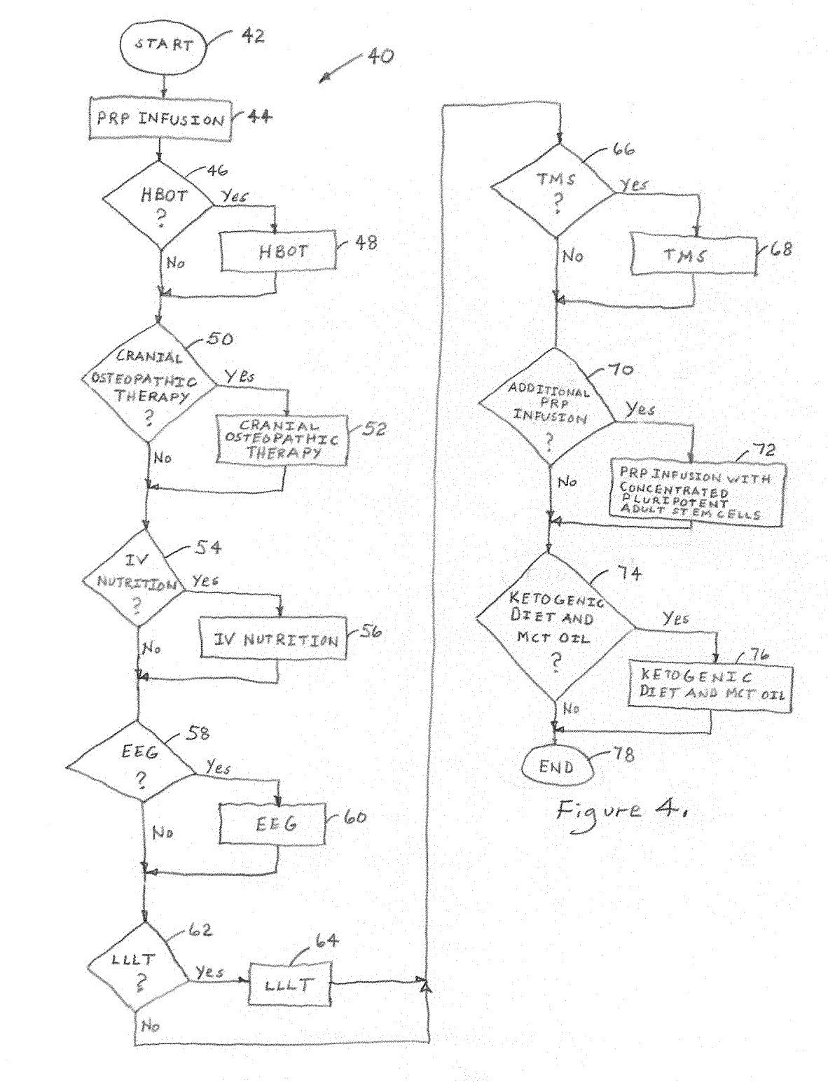

A non-limiting example of a method in accordance with the present invention for treating a patient who has suffered traumatic brain injury, generally indicated by the numeral 40, is shown in FIG. 4. The treatment method commences with an initial consultation between a qualified physician and the patient, as indicated by a step 42. As indicated by a step 44, the patient is treated using PRP infusion consisting of the above-described formulation using the formulation delivery apparatus 30, 80, or 90 at the time of the initial consultation. The step 44 is then preferably repeated a second time within 24 hours and may also be repeated every three weeks for a total of three additional treatments over six weeks. The PRP infusion therapy also be continued every four months for the following year.

In accordance with an additional non-limiting example of the treatment method of the present invention, a regimen of one or more treatments using hyperbaric oxygen therapy (HBOT) is administered by the qualified physician to the patient who has experienced traumatic brain injury, as indicated by the “Yes” branch from a decision block 46. During the period of administration of the HBOT treatments, as indicated by a step 48, the patient may also be treated using PRP infusion one or more times as described above. Additionally, following each treatment of PRP infusion, in accordance with a further non-limiting example of the treatment method of the present invention, cranial osteopathic therapy is received by the patient, as indicated by the “Yes” branch from a decision block 50, and performed on the patient by the qualified physician, as indicated by a step 52.

In accordance with a further non-limiting example of the present invention, a regimen of, for example, 40 treatments using hyperbaric oxygen therapy (HBOT) at the step 48 may be administered by a qualified physician to the patient, who has experienced brain trauma, over a period of six weeks. During the six-week period of administration of the HBOT treatments at the step 48, the patient may be treated using PRP infusion at the step 44 at the time of the first HBOT treatment, at three weeks, and also at six weeks. Following each treatment using PRP infusion initially and again at three and six weeks, cranial osteopathic therapy at the step 52 is performed on the patient. By way of an additional non-limiting example of the treatment method in accordance with the present invention, cranial osteopathic therapy at the step 52 may be performed on the patient three days a week for the first six weeks, then two days a week for one month thereafter, and then one day a week for the remainder of the period of one year from the initial consultation.

In accordance with another non-limiting example of the present invention, a regimen of 40 initial treatments using hyperbaric oxygen therapy (HBOT) at the step 48 administered by a qualified physician to a patient, who has experienced brain trauma, over a period of six weeks may be supplemented by two follow-up regimens of 20 treatments using HBOT at three and nine months from the initial HBOT regimen. The patient may be treated using supplemental PRP infusions at the step 44 at four-month intervals for a year from the time that the initial HBOT treatment is administered at the step 48. Following each supplemental treatment using PRP infusion initially and again at four-month intervals for a year from the time that the initial HBOT treatment is administered at the step 48, cranial osteopathic therapy is performed on the patient at the step 52. By way of an additional non-limiting example of the treatment method in accordance with the present invention, cranial osteopathic therapy at the step 52 may be performed on the patient three days a week for the first six weeks, then two days a week for one month thereafter, and then one day a week for the remainder of the period of one year from the initial consultation.

Cranial osteopathic techniques are employed to enhance delivery of intranasal plasma to the brain. Cranial osteopathy moves particular bones, including the ethmoid, vomer, frontal, sphenoid, temporal, and parietal, to maximize uptake into the brain tissue largely because of improvement in CSF flow and removal of osseous blockages to the olfactory nerve and trigeminal nerve pathways to the brain. Additionally, patients may also receive cranial facial release treatments before intranasal PRP administration, which release impingements around the ethmoid, maxillary, sphenoid, and frontal sinuses and associated bones.

In accordance with additional non-limiting examples of the present invention, PRP infusion, HBOT, and cranial osteopathic therapy may be further supplemented with additional therapies. The additional supplemental therapies may include one or more of the following therapies: intravenous (IV) nutrition, electroencephalographic (EEG) biofeedback, low level laser therapy (LLLT), and transcranial magnetic stimulation (TMS) treatments.

As shown in FIG. 4, in accordance with a further non-limiting example of the treatment method of the present invention, the patient may receive IV nutrition therapy, as indicated by the “Yes” branch from a decision block 54, and performed on the patient by the qualified physician, as indicated by a step 56. IV nutrition therapy is recommended three times a week while the patient is receiving HBOT. The HBOT potentizes the nutrients used for the IV nutritional therapy, so that the nutrients are made more bioavailable to the brain and other nervous system tissues.

As also shown in FIG. 4, in accordance with a still further non-limiting example of the treatment method of the present invention, the patient may be trained using EEG biofeedback, as indicated by the “Yes” branch from a decision block 58, and performed on the patient, as indicated by a step 60. For example, 40 initial EEG biofeedback treatments may be recommended at the times that hyperbaric oxygen therapy (HBOT) is administered at the step 48 with 10 follow-up sessions of EEG biofeedback at the step 60 performed every two months for one year after the initial 40 sessions.

As additionally shown in FIG. 4, in accordance with yet a further non-limiting example of the treatment method of the present invention, the patient may be treated using LLLT, as indicated by the “Yes” branch from a decision block 62, and performed on the patient, as indicated by a step 64. The LLLT therapy reduces inflammatory mediators such as prostaglandins, decreases bleeding, and improves mitochondrial ATP production. LLLT has been shown to penetrate the skull to directly aid damaged neurological tissue. LLLT therapy may be performed daily for the first six weeks from the initial consultation at the step 42 and preferably immediately before or after HBOT is administered. Follow-up treatments using LLLT are also preferably performed again at the above-described three and nine month intervals at which HBOT is preferably administered totaling 60 treatments.

As further shown in FIG. 4, in accordance with another non-limiting example of the treatment method of the present invention, the patient may be treated using TMS therapy, as indicated by the “Yes” branch from a decision block 66, and performed on the patient, as indicated by a step 68. TMS therapy employs an electromagnetic field that causes depolarization of the neurons in the brain, which has been theorized to aid connections in the brain and improve the emotional aspects of the functioning of the human brain. TMS therapy may be performed daily for the first six weeks from the initial consultation at the step 42 and followed up with 20 additional sessions for a total of 50 treatments.

As also shown in FIG. 4, in accordance with a further non-limiting example of the treatment method of the present invention, patients preferably receive at least a second PRP infusion with concentrated pluripotent adult stem cells, as indicated by the “Yes” branch from a decision block 70, and performed on the patient, as indicated by a step 72. The PRP concentrated pluripotent adult stem cells comprising the blood plasma of the patient are preferably infused intranasally and by IV. The procedure can be performed in approximately three hours and has proven to be very safe. Preferably, at least two PRP pluripotent adult stem cell treatments are recommended, for example, one treatment at the time of the initial consultation at the step 42 and a second treatment within 24 hours at the step 72.

As further shown in FIG. 4, in accordance with another non-limiting example of the treatment method of the present invention, the patient may be treated using a ketogenic diet and medium-chain triglyceride (MCT) oil therapy, as indicated by the “Yes” branch from a decision block 74, as indicated by a step 76. The ketogenic diet and MCT oil produce an associated modification of the tricarboxylic acid cycle to increase the synthesis of the neurotransmitter gammaaminobutyric acid (GABA), leading to neuronal hyperpolarization and less of the neuroxcitatory glutamate. A ketogenic diet and MCT oil provide improved protection from oxidative stress and increased synthesis of calming neurotransmitters (including GABA). As shown in FIG. 4, the treatment method 40 concludes at a step 78.

The following are examples of Case Reports demonstrating the efficacy of treatment of traumatic brain injury patients using the formulation described above infused using the formulation delivery apparatus 30, 80, or 90.

The following Case Report chronicles a male patient with a traumatic brain injury (TBI) due to a serious motor vehicle accident in August 2012, during which he experienced a direct blow to the frontal region of the head and brain. This patient further experienced significant post-concussion symptoms secondary to the TBI, including insomnia, photosensitivity, hyperacusis, memory loss, decreased ability to concentrate, emotional distress, depression, loss of libido, daily headache pain, and loss of executive function, as well as other related symptoms.

It has been found that a protocol utilizing multiple modalities over a three-to-nine month period is an effective way to treat sub-acute and chronic traumatic brain injuries. This protocol is not limited to, but may include, hyperbaric oxygen therapy (HBOT), human plasma composed of pluripotent adult stem cells, and cranial osteopathic therapy along with the adjunctive therapies of EEG biofeedback, IV nutritional therapy, transcranial magnetic stimulation (TMS), and low-level light therapy. Although several modalities in the protocol have been utilized singularly, a combination of these therapies in a synergistic manner yields a novel approach towards the long-term remediation of TBI. Also, particularly unique to the protocol is the administration of activated plasma (in a solution of nutrients and drugs), as well as plasma-derived pluripotent adult stem cells, directly into the frontal area of the brain.

By way of background, on Aug. 28, 2012, the patient, who will hereafter be referred to as Mr. Chad, a 46-year-old male, was involved in a motor vehicle accident in which his automobile was struck at speed by another vehicle. Mr. Chad remembered his head hitting the visor and possibly the windshield before he felt a twisted snap and then “blacked out” and was taken to the emergency room. His right eye had “popped out” of its socket. Mr. Chad had left-sided numbness for a while with tingling into his left arm. He also experienced pain in his neck, left sacroiliac joint, and left lower extremity.

Mr. Chad had been evaluated by many specialists and therapists since the motor vehicle accident in August 2012. He reported gaining benefit from stem cell and plasma (as PRP) injections into the spinal region around his neck. Mr. Chad also reported a reduction in musculo-skeletal pain over time since the injury with use of a home HBOT chamber and intramuscular “stem-cell” injections at a clinic in Boulder, Colo. Mr. Chad reported that he had experienced some relief of symptoms in the late spring of 2013, with the daily use of a home hyperbaric oxygen chamber and five sessions of EEG biofeedback in Boulder, Colo. He also received an initial evaluation and treatment with neurology by a neuropsychologist of the Centeno-Schultz Clinic in Boulder, Colo. However, he continued to experience significant symptoms 11 months after the motor vehicle accident in August 2012.

Mr. Chad's primary concerns upon first presentation on Jul. 15, 2013 were the symptoms from his head injury. He reported extreme sound and light sensitivity, as well as an inability to do math or other focused exercises such as reading. Mr. Chad also reported bouts of depression, anxiety, and physical and mental fatigue. He reported memory loss, space and time recognition, loss of libido, inability to carry on conversations, as well as daily, continuous headaches. Mr. Chad also reported living in darkness, being able to only “withstand five seconds of sunlight.”

At the time of intake, Mr. Chad denied current medications but reported using amino acids, 5 HTP, and a supplement known as Neuroreplete. He reported an allergic reaction to Codeine. His past medical history included a tracheal cyst and stomach ulcer. Mr. Chad's family history is noncontributory. He denied the use of alcohol, tobacco, and drug abuse (with the exception of medical cannabis for headache pain). He lives alone in a warehouse.

Mr. Chad's review of his systems was significant for the following. He reported daily headaches. He reported trouble with temperature changes. He reported loss of peripheral vision that had improved since he started using the hyperbaric oxygen chamber at home. He reported seeing dark spots in his vision. He reported hyperacusis. He reported constant neck pain and stiffness. He reported indigestion and reflux. He reported pain in his lower back and sacroiliac area. He reported right knee pain secondary to hitting on the dashboard in the motor vehicle accident. He reported having no medial collateral ligament. He reported having trouble initiating words, actions; trouble following through with plans; trouble with concentration. He reported depression, anxiety, and insomnia.

At his physical examination on Jul. 15, 2013, Mr. Chad had stable vital signs and presented with no acute distress. He was tender to palpation in the frontal area and temporal areas of his skull. He was wearing sunglasses and noise-canceling large headphones. He was able to hear in both ears. He had loss of vision peripherally, particularly significant in the right lower quadrant. His neck had a loss of range of motion, and he was most tender to palpation at the C5-C6 vertebral region bilaterally to the spine as well as C0 at the splenius capitus attachments. His lungs were clear to auscultation. His heart had a resting rate and regular rhythm with audible S1, S2 and no murmurs and no clicks. His abdomen was soft, with mild tenderness to palpation in the mid-epigastric area. He was mildly obese. His upper back was tender to palpation at the bilateral rhomboid attachments near the vertebral region of T7-T8. He was tender to palpation at the left L5-S1 lumbro-sacral ligaments. He had tenderness to palpation at the left SI ligament. His upper extremity and lower extremity reflexes were intact and 2/4 bilaterally. He had tenderness to palpation at the right medial knee. He had decreased grip strength of 3/5 in his left hand. There were no obvious skin lesions. His affect was mildly depressed, but he expressed some hope based on his treatments using the hyperbaric oxygen chamber at home.

Mr. Chad's MRI study of his neck (dated Oct. 11, 2012) demonstrated a left-sided cervical disc extrusion at C5-C6 on the left. Mr. Chad's MRI study of the brain with and without contrast (dated Oct. 11, 2012) was unremarkable for bleeding, mass, or appreciate insult to any area of the brain. Neither CT-scan nor PET/CT imaging studies had been performed on Mr. Chad.

Mr. Chad was still significantly mentally impaired when he presented on Jul. 15, 2013 in Basalt, Colo. At that time, Mr. Chad received an evaluation for commercialized HBOT therapy for TBI. Mr. Chad's plan was HBOT treatments at the standard TBI protocol of 1.5 atmospheres for 40 sessions. He was to undergo a neuropsychiatric evaluation to be completed in one week. He was to report about his sciatic pain and neck pain in two-to-three weeks. He was advised to take a baby aspirin daily and continue long-term follow-on treatment with neuropsychology, neurology, and physical therapy and to consider manual therapy to this head.

After an initial 25 sessions of HBOT at 1.5 atmospheres, cranial osteopathic therapy was performed on Mr. Chad, and activated plasma was administered to Mr. Chad in the form of injections, intravenous administration, and an intranasal drip. Mr. Chad also was given IV nutritional therapy to assist with his healing and recovery. He received 25 more HBOT treatments until the end of September 2013. The details of Mr. Chad's course of treatment are as follows.

From Jul. 15, 2013 to October 2013, Mr. Chad was treated with HBOT for 50 sessions at variable pressures ranging from 1.5 to 2.9 atmospheres. He received activated plasma injections on Aug. 26, 2013. He received activated plasma injections in the neck (composed of autologous human plasma, dextrose 5%, and calcium chloride (½ cc)) and activated plasma infusions (composed of autologous human plasma, dextrose 5%, 1 cc glutathione (200 mg/cc), ½ cc methylcobalamin (5000 mcg/cc), 20 units of insulin, and 4 cc O3 at 12 ug/cc) intranasally via pipette to the cribriform fossa. Mr. Chad received IV nutritional therapy two times over the course of three months. He received cranial osteopathic therapy eight times over the course of 12 weeks.

Improvements on physical examination and neuropsychiatric testing formed the primary assessment tool for understanding Mr. Chad's condition and effectiveness of his treatment regimen. Physical examination findings in April 2014 (approximately nine months after treatment initiation in July 2013) included diminished light and sound sensitivity, decreased tenderness in affected musculo-skeletal areas, reduced fatigue, and calm affect.

From July 2013 to October 2013, Mr. Chad made significant, rapid improvements in cognition, executive function, emotional affect, insomnia, fatigue, fear, and pain along with having a decrease in light and sound sensitivity.

In October 2013, Mr. Chad was able to fly on an airplane to Miami, Fla. after only three months of treatment with HBOT, activated plasma, IV nutritional therapy, and cranial osteopathic therapy. In April 2014, Mr. Chad demonstrated continued improvement and stabilization of his mental state. His neuropsychiatric evaluation also showed improvements. Third party follow-up post-treatment evaluations by a neuropsychologist in Boulder, Colo. also demonstrated improvements. Because Mr. Chad's traumatic brain injury was more neuropsychological than purely neurological, MRI and CT scans were not relied upon to determine significant effects of the protocol. It is noted that Mr. Chad had “fraying of his spinal cord” in the thoracic area as well as a cervical disc extrusion upon initial MRI but no major defects on MRI of the brain were observed upon initial presentation in July 2013. The assessment on Jul. 15, 2013 was moderate TBI with post-concussion syndrome with a wide neurological array of symptoms. He had cervical disc extrusion with possible radiculopathy into his left hand. He had sciatica, vision loss, and daily headaches.

From physical examination evidence and neuropsychiatric testing, Mr. Chad, a surviving TBI patient, made significant improvements in his mental capabilities and psychological response to the core treatment protocol involving HBOT, human plasma composed of pluripotent adult stem cells, and cranial osteopathic therapy along with the adjunctive therapies of IV nutritional therapy and EEG biofeedback. Mr. Chad's results demonstrate improvements intellectually, physiologically, and psychologically along with mood and personality improvements. Improvements in Mr. Chad's brain condition and symptoms have been verified by an independent neuropsychologist in Boulder, Colo. Other patients with mild to moderate traumatic brain injuries have also benefited from a similar treatment protocol with reported findings of “a 37% increase in my Lumosity score, less crying, and more timed thoughts.” Another mild-TBI patient who has completed a limited protocol reported that “my mind is more clear than it has been in years . . . I am much closer to the vitality and clarity that I used to feel in my mind every day.”

It is clear that Mr. Chad's Case Report demonstrates benefit of a treatment protocol with HBOT, human plasma composed of pluripotent adult stem cells, and cranial osteopathic therapy (including the intranasal delivery of the autologous human plasma, nutrient, and insulin) for patients with mild to moderate traumatic brain injury. The utilization of EEG readings, PET scans, functional MRI scans, and more neuropsychological testing is warranted to further determine the full efficacy of the TBI treatment protocol.

The following is a Case Report for a mild TBI patient treated with intermittent home hyperbaric oxygen therapy (HBOT), an intranasal and IV PRP formulation comprising pluripotent adult stem cells from peripheral blood, intranasal insulin, cranial osteopathy, and a ketogenic diet Ms. Pan suffered post concussive symptoms after hitting her head in December 2016.

Physical Findings and Assessment

### History and Exam on Feb. 9, 2017

Ms. Pan is a 28-year-old female who reported hitting her head on a toilet while traveling in a foreign country. The patient reported hitting her head when in the ladies restroom; she remembered standing up and feeling a little dizzy before hitting her head on the toilet and losing consciousness briefly. She reported pain on the top right side of her head. Ms. Pan performed several home hyperbaric oxygen therapy treatments in the past two weeks which seem to have helped her feel a bit better but continued to suffer from post concussive symptoms including headache pain, memory loss, difficulty making decisions, difficulty communicating thoughts and ideas, and sleeping (and staying asleep), as well as handling the stressors of everyday life.

REVIEW OF SYSTEMS: General; No weight change, generally healthy, feels physically and mentally fatigued daily; Head—Headache pain daily; Eyes—reports blurred vision since accident—comes and goes; Ears—No change in hearing, no tinnitus, no bleeding, no vertigo; Nose—No epistaxis, no coryza, no obstruction, no discharge; Mouth—No dental difficulties, no gingival bleeding, no use of dentures; Neck—reports stiff and achy neck; Chest—No dyspnea, no wheezing, no hemoptysis, no cough; Heart—No chest pains, no palpitations, no syncope, no orthopnea; Abdomen—No change in appetite, no dysphagia, no abdominal pains, no bowel habit changes, no emesis, no melena; Musculo-skeletal—Reports pain in right hip area; Neurologic—post concussive symptoms; Psychiatric—mood swings; trouble sleeping.

PHYSICAL EXAM FINDINGS: General: Normotensive, in no acute distress; Head—TTP over right parietal area—central zone; decreased CIU, venous sinus congestion; right side bending torsion; Eyes—PERRLA, EOM's full, conjunctivae clear, fundi grossly normal; Ears—EAC's clear, TM's normal; Nose—Mucosa normal, no obstruction; Throat—Clear, no exudates, no lesions; Neck—TTP at C2 bilat transverse processes; Chest—Lungs clear, no rales, no rhonchi, no wheezes; Heart—RR, no murmurs, no rubs, no gallops; Abdomen—Soft, no tenderness, no masses, BS normal; Back—Normal curvature, TTP at bilat scapular spine at trapezius/levator attachments; Extremities—decreased ROM of right hip flexor, TTP at right rectus femoris at origin; Neuro—post concussive findings; Psyche—increased affect, anxious.

ASSESSMENT: Ms. Pan was assessed with the following conditions:
TBI with post concussive syndrome, including headache pain, memory loss, difficulty making decisions, difficulty communicating thoughts and ideas, and sleeping (and staying asleep), mood swings, and ability to cope with stressors.
Whiplash
Cervicalgia
Upper back and neck strain
Right rectus femoris strain
Somatic dysfunction of Head, neck, back, and lower extremity

Management and Outcome

Ms. Pan was provided with the following recommendations:
TBI therapy protocol, including intranasal plasma formulation, IV nutrition, intranasal and IV pluripotent adult stem cells from peripheral blood, and intranasal insulin
Continue MCT oil, ketogenic diet, and nutritional support
Cranial osteopathy
Musculo-skeletal injections to neck and right LE
Continue home hyperbaric oxygen therapy treatment (referral to medical grade hyperbaric chamber if available)
Home stretching and PT

### Procedure Performed on Feb. 9, 2017

Ms. Pan was properly consented and understood risks of procedure. IV infusion of Myers nutrients was performed before procedure.

Using sterile technique, the patient was infused intranasally with a sterile solution (composed of a 0.3% ropivacaine, trace MgCl2)

Right nares (4 cc)
Left nares (4 cc)

Then the patient was infused with a sterile solution (composed of 1 cc D50, 7 cc autologous plasma, trace HCl, trace ascorbate, 40 units Humulin R, ¾ cc glutathione, ¼ cc B12):

Right nares (3 cc)
Left nares (3 cc)

The patient was stable after intranasal infusions and advised to rest, use ice, and over the counter medications for pain.

### Procedure Performed on Feb. 10, 2017

Intranasal infusion, IV, and injection of PBD-PSC treatment (platelet derived pluripotent adult stem cells) were performed. Ms. Pan was properly consented and understood the risks of the procedures.

Using sterile technique, the patient was infused intranasally with a sterile solution (composed of a 0.3% ropivacaine, trace MgCl2) with a nasal atomizer:

Right nares (3 cc)
Left nares (3 cc)

Then the patient was infused with a sterile solution composed of platelet derived autologous pluripotent adult stem cells (harvested and prepared in a sterile fashion diluted 50% with NS) with the specialized catheter 82:

Right nares (8 cc)
Left nares (8 cc)

The patient was then injected with a sterile solution (composed of 0.3% ropi, D10, trace MgCl2, trace ascorbate) followed by a sterile solution (composed of platelet derived autologous pluripotent adult stem cells (harvested and prepared in a sterile fashion diluted 50% with NS) into the following areas with a 25 g 2-inch needle:

Left scapular spine 2 cm lateral to L angle (3 cc ropi, 7 cc PBD-PSC)
Right scapular spine at R angle (3 cc ropi, 6 cc PBD-PSC)
C2 L IT ligament (3 cc ropi, 5 cc PBD-PSC)
C2 R IT ligament (3 cc ropi, 5 cc PBD-PSC)
Right rectus femoris at AIIS (4 cc ropi, 10 cc PBD-PSC)

8 cc of PBD-PSC formulation was infused intravenously into the patient's antecubital vein on the left arm.

A medical grade HBOT treatment to 1.75 atmospheres was also performed.

The patient was stable after injections and advised to rest, use ice, and over the counter medications for pain.

### Follow-Up on Feb. 10, 2017

Ms. Pan was seen again at the clinic and reported the following improvements:

Memory 30% improvement
Focus/concentration 40% improvement
Sleep 50% improvement
Light and sound sensitivity 75% improvement
Ability to handle stress 40% improvement
Moods 50% improvement
She reported, “I went from not being able to leave the house or work to being able to go outside, on walks and work part time from home.”
She also reported a 50-60% reduction in neck pain and 95-100% resolution of her right hip pain.

### CONCLUSIONS

Treatment for mild TBI by the multimodal application of hyperbaric oxygen therapy, PRP, pluripotent adult stem cells, intranasal insulin, and cranial osteopathy provides an effective solution to the post concussive problems faced by many TBI patients, particularly mild TBI patients. While this multimodal therapy may take 3-4 months for its full effects, many of these treatments can be applied over a 3-day period in a clinical setting and followed up by home use of an HBOT chamber by the patient.

In accordance with another non-limiting example of the present invention for the treatment of traumatic brain injury (or other neurological impairments or for neurological/cognitive enhancement), a further non-limiting example will now be described, comprising three stages of treatment, namely, TBI Therapy Pre-Treatment, Basic TBI Therapy Treatment, and TBI Therapy Post-Treatment.

TBI Therapy Pre-Treatment Protocol

The patient begins the following program three weeks before Basic TBI Therapy Treatment to be described below. Each treatment and supplement are recommended for optimal results.

I. Hyperbaric Oxygen Therapy (HBOT): 10-40 sessions in medical facility at 1.5 to 2.0 atmospheres for 60 minutes or Low Pressure Home Oxygen Therapy of at least 20 sessions in an in-home chamber at 1.3 atmospheres for 75 minutes.

II. Cranial Osteopathy: One session per week (if available).

III. EEG Biofeedback/Neurofeedback: One session per week (if available).

IV. Brainwave Training Player: 30 minutes a day (stabilizes brainwave patterns through the use of sound waves devised by Dr. Jeffrey Thomson).

V. Pretreatment Supplements: Begin 15 days before PRP treatment:
A. Stem XCell™ by Enzymedica: Two pills 2×/day. (Two bottles are needed throughout treatment. Stem XCell™ contains a university-researched blend, NT-020, consisting of Blueberry, Vitamin D, Green Tea Extract, and Carnosine. Scientific data suggests supplementation of NT-020 promotes the growth and health of stem cells, encouraging cell renewal.)
B. Brain Octane by Bulletproof: One tbsp. 2×/day with meals. (Three to four bottles are needed throughout treatment. Brain Octane is made with only C8 MCTs, which metabolize more efficiently into ketone energy than more common oils with C10 and C12 (Laurie Acid MCTs)).
C. Elk Antler by High Wire Ranch: Two pills 2×/day. (Four bottles are needed throughout treatment.)
D. E3 Live BrainON: One pill in AM; Two pills in PM. (One bottle is needed throughout treatment. Promotes mood balance, enhanced focus, ability to manage everyday stress, modulates neurotransmitters, and supports healthy inflammation responses).

Basic TBI Therapy Treatment Protocol

I. Hyperbaric Oxygen Therapy (HBOT): Angiogenesis, decreased inflammation, tissue regrowth, mobilization of stem cells, and increased metabolic activity.

II. Intranasal and Intravenous PRP: Regrowth of brain collagen, activation and targeting of pluripotent adult stem cells, increased angiogenesis, decreased inflammation (decreased cox 1, 2), reduction of b-amyloid proteins (that result in memory loss), increased brain glucose utilization, increased neurogenesis, and decreased cortisol.

III. Intranasal and Intravenous PRP-PBSC (Plasma Based Stem Cells): Regenerative and therapeutic properties, increased growth and healing factors, that initiate repair and attract the critical assistance of stem cells.

IV. IV Nutrition: Improved metabolic activity, improved detoxification, reduction of systemic pain and inflammation.

V. Cranial Osteopathy: Improved CSF flow, reduction of headache pain, and improved nourishment.

VI. Ketogenic Diet: Improved protection from oxidative stress and increased synthesis of calming neurotransmitters (including GABA).

Administration of Basic TBI Therapy Treatment Protocol:

Day One (1.5 hours in AM): Physician consultation and ketogenic diet discussion (60 min.), cranial osteopathy (30 min.), and HBOT (90 min.).

Day Two (2 hours in AM and 3 hours in PM): Blood draw (20 min.), intravenous PRP+IV Nutrition (60 min.), intranasal PRP (45 min.), HBOT (90 min.), intravenous stem cells+nicotinamide adenine dinucleotide+ (NAD+) (90 min.), intranasal PRP pluripotent adult stem cells (45 min.), and review supplements and take homes (30 min.).

TBI Therapy Post-Treatment Protocol

I. Hyperbaric Oxygen Therapy (HBOT): 40-50 sessions in a medical facility at 1.5-2.0 atmospheres total or 3-9 months of Low Pressure Home Oxygen Therapy at 1.3 atmospheres for 75-90 min., 5-7 days a week.

II. Cranial Osteopathy: One session per week for 12 weeks. The patient should continue cranial therapy with a local provider at least once a week or as their provider recommends.

III. Intranasal Insulin: 10-21 days every day for 10 days. If tolerated, it is recommended to continue use for three weeks, then take a one week break. If tolerated, the patient may repeat intranasal insulin for three weeks each month for three months.

IV. Brainwave Training Player: Listen to 30 minutes a day for 12 weeks. If the patient has abnormal mood changes while listening to the player, she or he should decrease to 1×/week and call her or his physician for further instructions.

V. Nutrition/Supplements:
A. Stem XCell™ by Enzymedica: Two pills 2×/day. Continue 15 days after PRP or PRP pluripotent adult stem cell treatment.
B. Brain Octane by Bulletproof: One tbsp. 2×/day with meals. Continue for 12 weeks.
C. Elk Antler by High Wire Ranch: Two pills 2×/day. Continue for eight weeks.
D. E3 Live BrainON: One pill in AM; two pills in PM. Finish one bottle.
E. Qualia by Neurohacker Collective: Only if recommended by the patient's physician.
F. Mexidol: Only if recommended by the patient's physician or two pills after drinking alcohol.

After the above-described three stages are completed, the following two additional phases of treatment may be performed.

Second Phase Treatment Protocol (3-6 months after initial treatment): As necessary (depending on severity of neurological condition), the patient repeats the Basic TBI Therapy Treatment Protocol described above. HBOT: The patient will receive a second set of HBOT treatments in a medical facility for 20 sessions at 1.5 to 2.0 atmospheres or continue using Low Pressure Home Oxygen Therapy at 1.3 atmospheres for 75-90 minutes, 5 days/week for 3 additional months. Cranial therapy is to be performed for one session a week for four weeks. The above-described Pretreatment Supplements are to be administered at least 15 days before the Second Phase Basic TBI Therapy Treatment Protocol commences.

Third Phase Treatment Protocol (6-12 months after initial treatment): As necessary (depending on severity of neurological condition), the patient repeats the Basic TBI Therapy Treatment Protocol. HBOT: The patient will receive a second set of HBOT treatments in a medical facility for 20 sessions at 1.5 to 2.0 atmospheres or continue using Low Pressure Home Oxygen Therapy at 1.3 atmospheres for 75 minutes for three months more. Cranial therapy is to be performed for one session a week for four weeks. The above-described Pretreatment Supplements are to be administered at least 15 days before the Third Phase Basic TBI Therapy Treatment Protocol commences.

While the foregoing description has been with reference to particular examples in accordance with the present invention, it will be appreciated by persons skilled in the art that changes in these examples may be made without departing from the principles and spirit of the invention. For example, the preferred parameters for the protocols in accordance with the various examples of the present invention described above are based on a sample of patients and may differ depending on the physical condition and characteristics of a given patient. Consequently, the parameters described for the various protocols are by way of example only and are to be considered to be within a range of the parameters that would apply to the general population as will be apparent to persons skilled in the art. Furthermore, in accordance with another non-limiting example of the present invention, a SphenoCath™ applicator available from Dolor Technologies, Inc. located in Salt Lake City, Utah may be used to infuse the formulations described above.

In addition to patients who have been treated for traumatic brain injury, patients whose brains have been injured with Reye's syndrome have also been successfully treated by the protocols in accordance with the various examples of the present invention described above. It is also believed that the protocols in accordance with the various examples of the present invention described above may be effective in the treatment of post-concussion syndrome (a mild, early form of brain injury) and chronic traumatic encephalopathy (CTE) and other later long-term consequences of untreated multiple brain injuries. Also, the administration of human PRP in accordance with the various examples of the present invention described above to treat stroke patients attenuates brain injury after focal ischemia following stroke. Furthermore, large, controlled studies, including level 1 evidence (which requires prospective examination and randomization), has led to a general acceptance that traumatic brain injury is a risk factor for developing Alzheimer's disease (AD). Accordingly, the protocols in accordance with the various examples of the present invention described above may be effective in reducing the incidence of AD. Accordingly, the various non-limiting examples described above provide novel treatment protocols for TBI and its post concussive symptoms, but are not limited to TBI patients because they also serve as effective treatments for patients with other neurological conditions, as well as for neuropsychological and cognitive enhancement. The medical indications that may benefit from the treatment protocols described above include the following: mild to severe traumatic, anoxic, or chemical/toxic brain injuries, neurodegenerative conditions (e.g., Alzheimer's disease, Parkinson's disease, MS, cerebral or cerebellar atropy, chronic traumatic encephalopathy (CTE), chronic inflammatory demyelinating polyneuropathy (CIDP), neurovascular conditions (e.g., embolic stroke), brain enhancement (including memory or cognitive enhancement), and brain longevity treatment. The scope of the present invention can only be ascertained with reference to the appended claims.

What is claimed is:

1. A formulation comprising platelet rich plasma (PRP) for treatment of patients who have experienced brain injury, consisting of: a mixture of human plasma composed of pluripotent adult stem cells comprising very small embryonic like stem cells from autologous plasma or allogeneic plasma; D5W; glutathione; methylcobalamin; and regular insulin.

2. A formulation as recited in claim 1 wherein the formulation consists of a mixture of approximately 75% by volume human plasma, 15% by volume D5W, 5% by volume glutathione, 4% by volume methylcobalamin, and less than 1% by volume regular insulin.

3. A formulation delivery apparatus, consisting of:
an intranasal delivery device, comprising:
a syringe having a reservoir and a dispensing tip; and
a catheter selectively attached to the dispensing tip of the syringe.

4. A formulation delivery apparatus as recited in claim 3 wherein the dispensing tip is an atomizing dispensing tip.

5. A formulation delivery apparatus as recited in claim 3 wherein the catheter is configured to be inserted into a naris of a brain injury patient, and the syringe is actuable with the catheter being positioned in the nasal cavity to provide delivery of a formulation to the fossa of the cribiform plate under the nasal bridge just posterior to the frontal bone of the cranium to infuse the formulation into the patient's brain.

6. A method for treatment of patients who have experienced brain injury, comprising slowly infusing a mixture of human plasma comprising pluripotent adult stem cells including very small embryonic like stem cells from autologous plasma or allogeneic plasma, D5W, glutathione, methylcobalamin, and regular insulin into the frontal region of the brain of a patient suffering from brain injury.

7. A method as recited in claim 6 wherein the mixture is delivered directly adjacent to the brain through the nostrils or nares of the nasal cavity.

8. A method as recited in claim 6 wherein infusion of the mixture is selectively repeated.

9. A method as recited in claim 7, further comprising:
administering to the patient a regimen of one or more treatments using hyperbaric oxygen therapy (HBOT).

10. A method as recited in claim 7, further comprising performing cranial osteopathic therapy on the patient.

11. A method as recited in claim 7, further comprising performing intravenous (IV) nutrition therapy on the patient.

12. A method as recited in claim 7, further comprising performing IV nutrition therapy on the patient.

13. A method as recited in claim 7, further comprising performing EEG biofeedback training on the patient.

14. A method as recited in claim 7, further comprising treating the patient using low level laser therapy (LLLT).

15. A method as recited in claim 7, further comprising treating the patient using transcranial magnetic stimulation (TMS) therapy.

16. A method as recited in claim 7, further comprising administering to the patient a ketogenic diet and medium-chain triglyceride (MCT) oil therapy.

17. A method as recited in claim 6, wherein the human plasma, comprising plasma based pluripotent adult stem cells, is concentrated by PRP growth factors combined with NAD+.

18. A method as recited in claim 17 wherein the mixture is activated by ozone and a green laser light at a specific frequency.
